(19) United States
(12) Patent Application Publication
(10) Pub. No.: US 2006/0020173 A1
(43) Pub. Date: Jan. 26, 2006

(54) Rib cage retractor and pads thereof

(76) Inventor: Marcel Gagne, St. Georges-De-Beauce (CA)

Correspondence Address: INVENTARIUM;Suite 1607, 4050 Rosemont Blvd, Montreal, H1X 1M4, CA

(21) Appl. No.: 11/128,666

(22) Filed: May 12, 2005

Related U.S. Application Data
(60) Provisional application No. 60/589,056, filed on Jul. 20, 2004.

(51) Int. Cl. A61B 1/32

(52) U.S. Cl. 600/219

(57) ABSTRACT

A rib cage retractor for game hunters comprises a bar clamp/spreader as is known in the art, such a tool has an end piece which is removable and that, when placed on the opposite end of the bar, acts as a spreader. Since the typical bar clamp is generally aimed at carpentry projects, it has been found necessary to modify the pads of the clamping means with more appropriate pads. The modified pads generally comprise a fork engageable into the split sternum of a cut ribcage. Optionally, a light source to light the interior of the rib cage, as is found in some of the prior art, can be incorporated in the ratcheting handle.

This application claims priority based on provisional application 60/589,056 filed Jul. 20, 2004 Rib cage retractor and pads thereof

### BACKGROUND OF THE INVENTION

1. Field of the Invention

This invention relates generally to game hunting equipment but more particularly to a tool that spreads open and maintains an open configuration to the rib cage of a dead game.

2. Background of the Invention

Tools designed to open the rib cage of humans for the purpose of surgery have been known for years, for example, U.S. Pat. No. 2,642,862 shows an early example of a surgical retractor which is good for holding the rib cage open but requires the strength of the surgeon to open it in the first place.

U.S. Pat. No. 3,196,865 requires a pair of such devices to provide retracting on each sides, each element containing a pluratlity of small components that favor proliferation of germs and bacteria.

U.S. Pat. No. 4,337,763 is provided with means for illuminating the interior the body cavity.

U.S. Pat. No. 4,627,421 shows a retractor with pivoting arms which reduces the amount of pressure exerted on the small ribs of the upper part of the rib cage when the rib cage is retracted for open heart surgery. A cursor moves along a cross-bar such that as the cursor is moved along the cross-bar, the opening will be retracted in a triangular shape.

U.S. Pat. No. 4,726,356 shows a cardiovascular retractor which is adapted to be mounted on a thoracic retractor. The cardiovascular retractor includes a clamp support which is mounted along an axis of a thoracic retractor and a retractor blade clamp adjustably secured to a rod of the clamp support. The clamp has a blade grip which grasps a retractor blade.

U.S. Pat. No. 5,067,477 shows a low wear bearing system for a surgical retractor including usually four bearings recessed in the retractor housing, each bearing having a generally rectangular projection extending into a corresponding slot in the rack of the rack and pinion type retractor.

U.S. Pat. No. 5,088,472 shows a sternal retractor comprising a pair of arms with sternum engaging blades thereon and a curved cross bar on which said arms are disposed such that in use the retractor can open the bottom of the sternum more than the top of the sternum to minimize damage and injury to the upper ribs and numbness which sometimes results in the hands of open chest surgery patients.

U.S. Pat. No. 5,897,490 shows an apparatus for pivoting a surgical retractor having two arms. The apparatus has a bridge for engaging at least one arm of the surgical retractor. The bridge has a distal end, a proximal end and a longitudinal axis extending therebetween. An arm extender comprising a bridge coupling and a blade is attached to the bridge and engages with the tissue of the surgical patient. The apparatus also has a lifting assembly comprising an elevator. The lifting assembly is for applying an upward force to the proximal end of the bridge.

U.S. Pat. No. 5,908,382 shows an intermammary harvesting retractor comprised of a retractor frame having a fixed or stationary arm on one end of a crossbar and a movable arm mounted on the crossbar by a crank mechanism. The crank mechanism allows the movable arm to be adjusted toward or away from the stationary arm. A support tower is mounted on the free end of the retractor crossbar to allow the angle of the retractor to be adjusted after placement in an incision. Adjustable lifter blades are provided on the movable arm to also adjust the lift angle of the upper ribcage.

U.S. Pat. No. 6,099,468 shows a retractor for use in partial sternotomy and includes a toothed crossbar to which a pair of small, parallel grips are attached. The first grip is attached to an arm which is fixed to one end of the crossbar. The second grip is attached to an arm which is movably attached to the crossbar by a pinion, thereby permitting the second grip to be moved along the crossbar toward or away from the first grip. A first rod is connected to the fixed arm by means of a bracket, while a second rod is connected to the movable arm by means of a similar bracket.

U.S. Pat. No. 6,331,158 shows a methods and apparatus for accessing and stabilizing the heart, it allows various instruments or devices to be maneuvered and secured in place, and provide stabilization of the heart.

These tools are generally quite expensive and are well beyond the means of the average game hunter. Moreover, the needs of the game hunter are quite different than the needs of the surgeon, so substantially different methods for opening a rib cage can be used for the needs of game hunters. There is therefore a need for an inexpensive, safe, and practical tool for opening game rib cages.

### SUMMARY OF THE INVENTION

In view of the foregoing, there does not appear to be a device responding to the needs of game hunters. Therefore, the present invention, which will be described subsequently in greater detail, is to provide objects and advantages addressed to the needs of game hunters and which are:

To provide an inexpensive rib cage retractor.

To provide for a simple to use rib cage retractor.

To attain these ends, the present invention uses a bar clamp/spreader as is known in the art, such a tool has an end piece which is removable and that, when placed on the opposite end of the bar, acts as a spreader. Since the typical bar clamp is generally aimed at carpentry projects, it has been found necessary to modify the pads of the clamping means with more appropriate pads. The modified pads generally comprise a fork engageable into the split sternum of a cut ribcage. Optionally, a light source to light the interior of the rib cage, as is found in some of the prior art, can be incorporated in the ratcheting handle.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

After a game has been killed, it is sometime preferable to empty its guts as quickly as possible. In order to efficiently eviscerate the game with minimal mess and soilage to oneself or the surroundings, it is preferable that the rib cage of the game be open and kept open to facilitate the operation. Usually, one or more fellow hunters take on the task but it is far from the ideal mode of operation since holding a rib cage wide open for a period of time can become tiresome.

### BRIEF DESCRIPTION OF THE PREFERRED EMBODIMENT

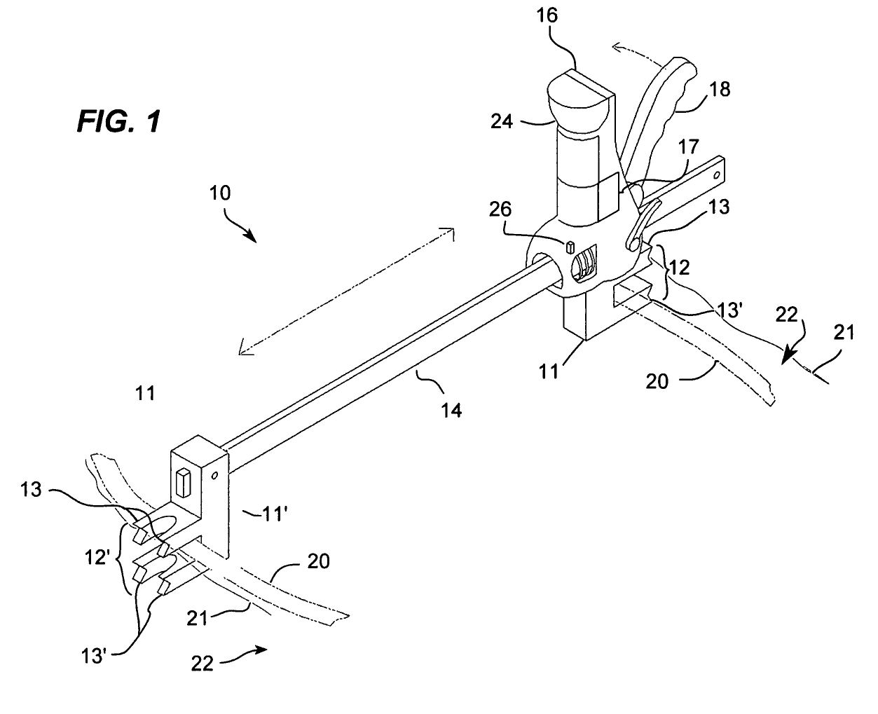

FIG. 1 Perspective view of the rib cage retractor.

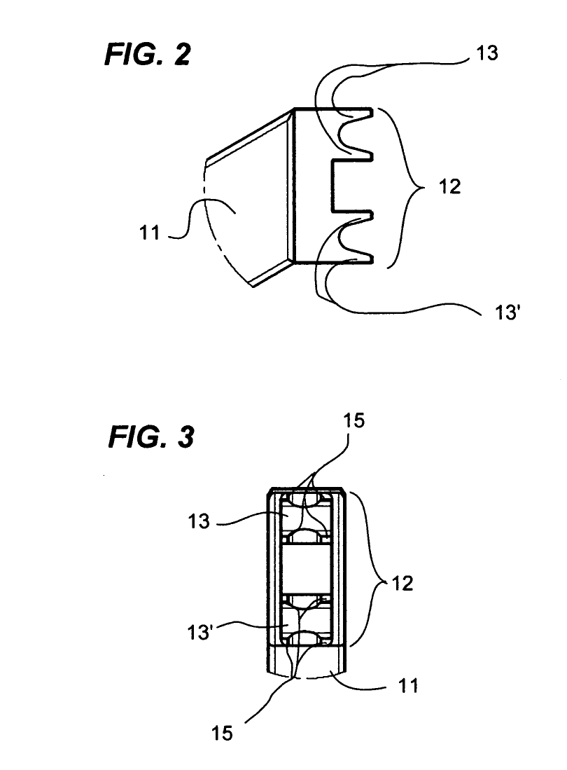

FIG. 2 Side view of a fork.

FIG. 3 Front view of a fork.

### DETAILED DESCRIPTION OF THE PREFERRED EMBODIMENT

A rib cage retractor (10) has two retracting means (11, 11′) set along a rod (14). A ratchet handle (16) slidingly engaging the rod (14) has one retracting means (11) extending integrally therefrom and has a trigger (18) which acts as separating means for separating each retracting means (11, 11′). The ratchet handle (16) slides along the rod (14) when actuated by the trigger (18) and thus incrementally separates the retracting means (11, 11′). A release tab (20) allows for free motion of the ratchet handle (16) along the rod (14).

The retracting means (11, 11′) are terminated by pads known as forks (12, 12′) which can be, as in the illustrated examples, part of the retracting means (11, 11′) or they can be separate components substituted to existing pads found on currently available bar clamps. The forks (12, 12′) are inserted into the split sternum (20) of a cut rib cage (22) and the rib cage retractor (10) is engaged in such a way that the ratchet handle (16) is on the outside of the rib cage (22). Actuating the trigger (18) moves one fork (12) along the rod (14) farther apart from the other fork (12′) and thus spreads open the rib cage (22). The forks (12, 12′) have preferably two prongs (13, 13′) with one prong (13) being on the exterior side of the rib cage (22), pushing flesh (21), while the other prong (13′) is on the interior side of the rib cage (22). Preferably the prongs (13, 13′) are set between ½ inch to 3 inches apart, center to center. Each prong (13, 13′) is itself made out of 4 corner points (15) used for pricking the flesh. In the illustrated example of FIGS. 2-3, the number of points (15) is four but it should be understood that there could be at least one point (15), two, three, or more than four such points (15) all within the scope of this invention. Each point (15) is between % inch long to 2 inch long. Each point (15) is separated by the next one by a generally <<U>> shaped space. The range in dimension is to accomodate the size of various game.

Optionally, a light source (24) with a switch (26) can be integrated into the ratchet handle (16) to provide illumination towards the interior of an animal (not shown). A battery access panel (17) is made into the handle (16).

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A rib cage retractor having two retracting means set along a rod; a ratchet handle slidingly engaging said rod; one retracting means extending integrally from said ratchet handle; said ratchet handle having a trigger for separating each retracting means; a release tab allowing for free motion of said ratchet handle along said rod and having said retracting means terminated by forks and; said forks having prongs.

2. A rib cage retractor having the following method of use:
forks are engaged into a split sternum of a cut rib cage;
a trigger, part of a handle is actuated so as to separate said forks and thus open said rib cage.

3. A rib cage retractor as in claim 1 wherein:
a light source is integrated into said ratchet handle to provide illumination inside the animal.

4. A rib cage retractor as in claim 1 wherein:
said forks being separate components substituted to existing pads found on currently available bar clamps.

5. A rib cage retractor as in claim 1 wherein:
said forks have two prongs with one prong being on the interior side of said rib cage while the other prong is on the outside of said rib cage.

6. A rib cage retractor as in claim 5 wherein:
prongs are set between ½ inch to 3 inches apart, center to center.

7. A rib cage retractor as in claim 5 wherein:
each prong being itself made out of 4 corner points.

8. A rib cage retractor as in claim 5 wherein:
each point from said prong is between ¼ inch long to 2 inch long.
